(19) United States
(12) Patent Application Publication
(10) Pub. No.: US 2003/0074044 A1
(43) Pub. Date: Apr. 17, 2003

(54) Stent delivery system

(76) Inventors: John H. Randby, Eagan, MN (US); Fernando DiCaprio, St. Paul, MN (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 09/982,052

(22) Filed: Oct. 16, 2001

(51) Int. Cl. A61F 2/06

(52) U.S. Cl. 623/1.11

(57) ABSTRACT

A stent delivery system to facilitate introduction and placement of a stent, including a catheter having an expandable distal portion constructed and arranged for expanding the outer diameter of the catheter from a contracted state to an expanded state. The stent is positioned around the distal portion of the catheter and is thereby expanded. The system further comprises retaining sleeves which are attached to the catheter and abut the ends of the stent to prevent axial movement and to aid in the retention of the stent.

### BACKGROUND OF THE INVENTION

In typical PTCA procedures, a guiding catheter is percutaneously introduced into the cardiovascular system of a patient through a vessel and advanced through therein until the distal end thereof is at a desired location in the vasculature. A guidewire and a dilatation catheter having a balloon on the distal end thereof are introduced through the guiding catheter with the guidewire sliding through the dilatation catheter. The guidewire is first advanced out of the guiding catheter into the patient's coronary vasculature and the dilatation catheter is advanced over the previously advanced guidewire until the dilatation balloon is properly positioned across the lesion. Once in position across the lesion, the flexible, expandable, preformed balloon is inflated to a predetermined size with a liquid or gas at relatively high pressures, such as greater than about four atmospheres, to radially compress the arthrosclerotic plaque of the lesion against the inside of the artery wall and thereby dilate the lumen of the artery. The balloon is then deflated to a small profile so that the dilatation catheter may be withdrawn from the patients vasculature and blood flow resumed through the dilated artery.

In angioplasty procedures of the kind described above, there may be injury to or restenosis of the artery, which either necessitates another angioplasty procedure, a surgical by-pass operation, or some method of repairing or strengthening the area. To strengthen the area and help prevent restenosis, a physician can implant an intravascular prosthesis for maintaining vascular patency, commonly called a stent, inside the artery at the lesion. The stent is expanded to a larger diameter for placement in the vasculature, often by the balloon portion of the catheter. Stents delivered to a restricted coronary artery, expanded to a larger diameter by a balloon catheter, and left in place in the artery at the site of a dilated lesion are shown in U.S. Pat. No. 4,740,207 to Kreamer and U.S. Pat. No. 5,007,926 to Derbyshire, the content of which is incorporated herein by reference. Palmaz et al., 156 Radiology 73 (1985) and U.S. Pat. No. 4,733,665 describe introduction of a stent over a balloon catheter (incorporated herein by reference). Any type of stent may be used with the present invention. Exemplary stents are shown in U.S. Pat. No. 4,735,665; U.S. Pat. No. 4,950,227; EPO application 707 837 A1, U.S. Pat. No. 5,445,646, PCT Publication WO 0758216 and U.S. Pat. No. 5,972,018 All of these patents are incorporated herein by reference and are intended to be exemplary only and not limiting. Various materials including stainless steel, tantalum, shape memory alloys and plastic may be used.

The stent delivery apparatus and method may also utilize a self-expanding stent, which is well known in the art. A well known self-expanding stent is the woven braided stent disclosed in U.S. Pat. Nos. 4,655,771 (Wallsten); 4,954,126 (Wallsten) and 5,061,275 (Wallsten), although any type of self-expanding stent may be deployed using the inventive delivery system and method. The delivery system of the present invention may also be used to deliver a balloon expandable stent and may also deliver stent grafts, which are well known in the art.

The delivery systems for stents are generally comprised of catheters with the stent axially surrounding the distal end of the catheter. Typically, the stent is crimped on the balloon portion of the catheter. In many stent delivery catheters the stent is retained on the balloon catheter with a radially disposed sleeve or sheath which may be retracted or otherwise removed to release the stent. More recently stent delivery systems have included one or more stent retaining sleeves or socks disposed about the respective ends of the stent to hold and/or protect the ends of the stent in the reduced configuration on the delivery catheter.

Inflation expandable stent delivery and deployment assemblies are known to utilize restraining means that overlie the stent during delivery. U.S. Pat. No. 4,950,227 to Savin et al., relates to an inflation expandable stent delivery system in which a sleeve overlaps the distal or proximal margin (or both) of the stent during delivery. During inflation of the stent at the deployment site, the stent margins are freed of the protective sleeve(s). U.S. Pat. No. 5,403,341 to Solar, relates to a stent delivery and deployment assembly which uses retaining sheaths positioned about opposite ends of the compressed stent. The retaining sheaths of Solar are adapted to tear under pressure as the stent is radially expanded, thus releasing the stent from engagement with the sheaths. U.S. Pat. No. 5,108,416 to Ryan et al., describes a stent introducer system which uses one or two flexible end caps and an annular socket surrounding the balloon to position the stent during introduction to the deployment site.

Other patents which describe sleeves, and material used therefor, include Dusbabek et al. U.S. Pat. No. 5,968,069, issued Oct. 19, 1999, and Cornelius et al., U.S. Pat. No. 6,068,634, issued May 30, 2000.

All patents and applications and all other published documents mentioned anywhere in this application are incorporated herein by reference in their entirety.

The invention in various of its embodiment is summarized below. Additional details of the invention and/or additional embodiments of the invention may be found in the Detailed Description of the Invention below.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

### SUMMARY OF THE INVENTION

This invention concerns apparatus suitable for delivery of stents to body cavities. The stent prosthesis is formed of a generally tubular body, the diameter of which can be decreased or increased. Stents are particularly useful for permanently widening a vessel which is either in a narrowed state, or internally supporting a damaged vessel. The catheter is usually of the balloon catheter type in which the balloon is utilized to expand the stent, which is positioned over the balloon, to place it in a selected location in the body cavity. The present invention is particularly directed to improved arrangements for releasably securing the stent, particularly the ends thereof, and to prevent snagging of the stent ends and to facilitate delivery thereof. The stent is held in place on the catheter and kept from moving axially or flaring upward at its end(s) by means of at least one sleeve, or sock, preferably two, abutting the ends of the stent, the stent having been fitted to the catheter over the balloon, as by crimping.

In particular, the invention is directed to aiding, via the sock arrangements, in protecting the stent from deformation, damage or premature release during delivery intraluminally, as well as snagging during transportation. It is also a purpose of the present invention to provide for easier and smoother removal of the sock. The stent is formed to its lowest geometrical diameter when loaded. The sleeves aid in retaining the stent in place (unexpanded) with little or no relative movement between the ID of the stent and the OD of the balloon/catheter arrangement. The socks preferably are elastomeric and are adhered, or alternatively frictionally engaged, to the catheter. When the balloon under the stent is inflated, the sleeves separate from the ends of the stent, primarily falling away in an axial direction, resulting in the sleeves being pushed/pulled down the balloon cones to allow the stent to deploy. The sleeves may remain on the cones during expansion. During expansion of the balloon, the sleeves may also spring back and fold back over themselves. This may take the form of a mere overlap or an “S” or “Z” configuration.

Most stents which are deformed to a low diameter will increase in diameter somewhat after being deformed crimped (spring back or recoil). The sleeves aid in preventing spring back and add an additional means of retention, holding the stent between the sleeves. The engagement and placement of the sleeves aids the middle of the stent to deploy first. For certain stents that have a tendency to flair at the ends during expansion, having the middle portion expand first allows for an evening of the expansion of the stent.

The sleeves are not engaged “on” the ends of the stent, but may be in contact with the ends of the stent. The sleeves are engaged on the distal and proximal ends of the balloon. This too keeps the ends of the stent from expanding first and allows the middle of the stent to deploy first. However, the ends of the balloon with the sleeves may also be configured to expand first.

The positioning, size and profile of the sleeves also allow for improved re-wrap of the balloon. The design makes it easier to withdrawal the catheter into the guiding catheter. It further aids in withdrawing the balloon out of the deployed stent. Re-wrap of balloons is discussed in U.S. Pat. No. 5,226,880, U.S. Pat. No. 5,476,476 and U.S. Pat. No. 5,116,318. The lower profile allows for reuse of the balloon to re-dilate the stent or lesion after the primary inflation.

The present design further provides a smoother distal balloon region by providing a more level profile transition at the ends of the stent. This allows for better lesion entry and ease of movement of the catheter within the guiding catheter.

All the cited references listed in the present document, patents or otherwise, are herein incorporated by reference in their entirety.

### BRIEF DESCRIPTION OF THE FIGURES

FIG. 1 is a profile view, in longitudinal section, of the distal end portion of a balloon catheter having a stent fixed to the catheter by being crimped thereto over the balloon, the ends of the stent being adjacent to a first embodiment of the invention;

FIG. 2 is similar to FIG. 1 in which the stent has been expanded;

FIG. 3 is a view of a modification of the embodiment shown in FIGS. 1 and 2; and

FIG. 4 is similar to FIG. 3 in which the stent has been expanded;.

FIG. 5 is a view of a modification of the embodiment shown in FIGS. 1 and 2;

FIG. 6 is similar to FIG. 5 in which the stent has been expanded; and

FIGS. 7-10 are side views of various sleeve configurations.

### DESCRIPTION OF THE PREFERRED EMBODIMENTS

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, unless otherwise indicated, identical reference numerals used in different figures refer to the same component.

For the purposes of this disclosure, the term stent refers to stents, stent-grafts, grafts and other endoluminal prostheses whether self-expanding, balloon expandable, self-expanding and balloon expandable or otherwise expandable as are known in the art.

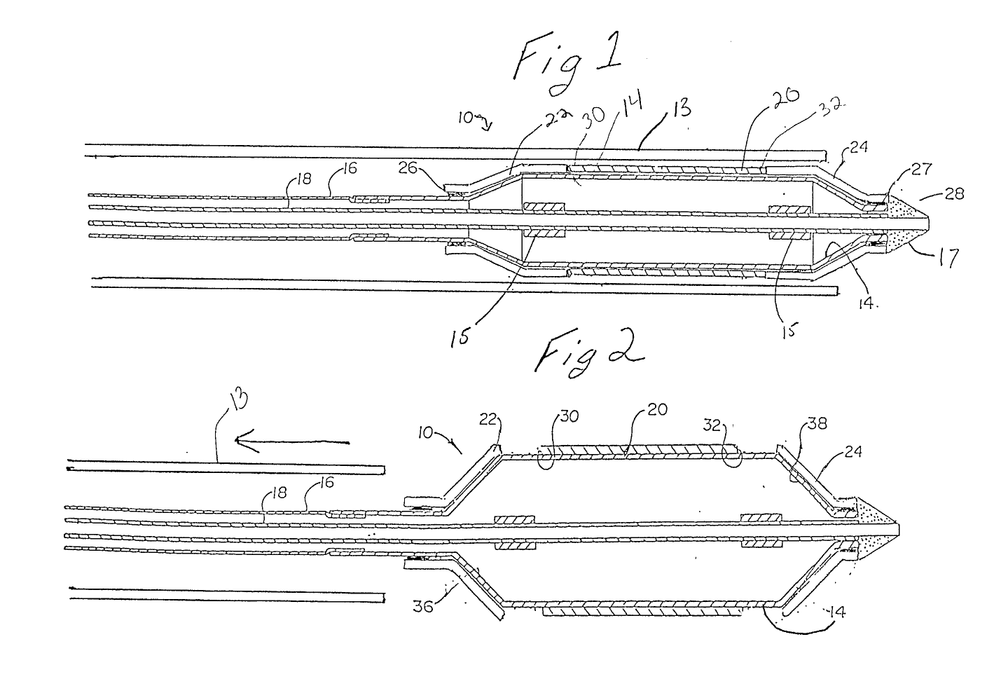

In addition to the over-the-wire embodiments (example also found in U.S. Pat. No. 5,980,533) shown in FIGS. 1-4, the inventive catheter system may also be provided in a rapid-exchange configuration. Examples of rapid-exchange catheters may be found in U.S. Pat. No. 5,534,007 and U.S. Pat. No. 5,833,706. The inventive stent delivery systems may also be made in fixed wire form. Examples of fixed-wire catheters may be found in U.S. Pat. No. 5,702,364.

The system may be adapted for use with a medical device such as a stent, for example, a self-expanding, balloon expandable or combination self-expanding and balloon expandable stent. The system may also be used for delivery of other medical devices for use in the body as well including, but not limited to, ultrasonic devices, laser devices, vena cava filters, implantable drug delivery devices and the like.

The inventive medical systems disclosed herein may also be provided with any of the features disclosed in U.S. Pat. No. 6,096,056, U.S. Pat. No. 6,068,634, U.S. Pat. No. 6,036,697, U.S. Pat. No. 6,007,543, U.S. Pat. No. 5,968,069, U.S. Pat. No. 5,957,930, U.S. Pat. No. 5,944,726, U.S. Pat. No. 5,653,691 and U.S. Pat. No. 5,534,007.

The stent delivery system may also comprise various coatings as are known in the art, including lubricious coatings to facilitate movement of the various parts of the system, as well as collagen-type coatings. More information concerning suitable coatings may be found in U.S. Pat. No. 5,443,907, and U.S. application Nos. 08/382478, 09/306939 and 09/316502.

The invention is also directed to medical device delivery systems and catheters produced using the inventive methods.

For the purposes of the detailed description of the invention, Figures of a portion of the distal end of a typical balloon catheter will be used. It should be understood, as mentioned above, that the present invention is applicable to other medical devices which require an expandable balloon. It should also be understood that the materials used may be any of those materials known in the art where applicable.

Referring to FIGS. 1 and 2, a stent delivery system 10 includes a catheter such as an over-the-wire, fixed wire or rapid exchange. Balloon catheters are preferred herein as best examples of catheters having an expandable distal end portion constructed and arranged for expanding the outer diameter of the catheter from a contracted state to an expanded state. FIGS. 1 and 2 show a catheter 10 having a guide catheter shaft 13, an outer shaft 16, a guide wire lumen 18 with marker bands 15 secured thereto and a distal tip 28. A balloon 14 is fixed to the distal end of the catheter, preferably by adhesive attachment of the proximal end to the outer shaft 16 of the catheter and the distal end to the inner shaft 18 of the catheter. Other arrangements known in the art may be used. Balloon 14 is shown in FIG. 1 in its contracted state and in FIG. 2 in its expanded state. A stent 20 is crimped about balloon 14 and by two adjacent retaining sleeves, 22 and 24, which abut the ends 30, 32 of the stent, respectively.

As mentioned above, various types of stents may be used with balloon expansion. For example, the stent may be a self-expanding stent which upon release self-expands and is further expanded or is merely aided in release by balloon expansion from the sleeves. Such stents may self-expand elastically or may be thermally induced such as stents formed of nitinol or other shape memory metals or materials.

Any kind of stent may be delivered by the system of the invention, including plastically deformable or elastically deformable and they may be of any configuration or structure so long as they can be loaded at a low diameter and deployed at a larger diameter, i.e., have a contracted condition and being expandable to an expanded condition of large diameter.

Stent 20 may be any of the various types known in the art, either balloon expandable or self-expandable. Exemplary stents are shown in U.S. Pat. No. 4,735,665; U.S. Pat. No. 4,950,227; EPO application 707 837 A1, and U.S. Pat. No. 5,445,646. All of these patents are incorporated herein by reference and are intended to be exemplary only and not limiting. Various materials including stainless steel, tantalum, shape memory alloys and plastic may be used.

Stent 20 is radially compressed, as by crimping to a contracted condition, against balloon 14 to a relatively small loaded diameter having an OD of approximately 0.025 to 0.08 inches for example, although it has a larger released diameter in the expanded condition. Various size stents may be used as dictated by their intended use.

Sleeves 22 and 24 may be formed of polyurethane tubing or the like, having for example an ID of 0.035-0.08 inches and a wall thickness of 0.002-0.006 inches, for example, and are axially attached along catheter 10 to the proximal end of balloon 14 at 26 and to the distal end of balloon 14 at 27 by means of polyurethane adhesive 26, 27. The sleeves also may be attached to the catheter and held in place by elastic tension, without any adhesive product or by thermal welding

The expandable material of the sleeves is preferably elastomers such as polyurethane, polyurethane blends, silicone, latex or polyether amide, by way of example only, most preferably polyolefin copolymer (POC); Surlyn™. The material should be formable into a thin walled tube. Although the invention contemplates embodiments having one sleeve at either end, the use of a pair of sleeves, one at each end of the stent, is preferred.

The sleeves may also be radiopaque. For such sleeves, radiopaque materials may be included on or within the sleeve. As such, the sleeves could replace, or work in tandem with, the marker bands 15. Radiopaque materials are well known in the industry.

Sleeves 22 and 24 abut stent 20 at each of its ends 30 and 32, respectively. They can be urged up against the ends to the stent, adding added securement of the stent without sacrificing overall profile, in that there is no overlap with the stent.

Referring to FIG. 2, in its expanded state balloon 14 has an enlarged diameter with tapered cone portions 36 and 38 at each end thereof. Stent 20 is released from engagement with sleeves 22 and 24 upon expansion of balloon 14, drawing the sleeves gradually outward. As the balloon is inflated, a combination of radial and axial forces are applied, expanding the stent and separating the ends of sleeves from the ends of the stent. The increased axial pressure allows for a more controlled and steady release of the stent and balloon. As seen in FIG. 2 the stent deploys. The sleeves contract about balloon 14 when it is deflated. Deflation allows balloon 14 and sleeves 22 and 24 along with catheter 10 to be axially removed from the body.

In assembling the polyurethane sleeves, they can be temporarily swelled by exposure to a solvent such as toluene, alcohol, or others as known in the art, then pushed on the ends of the stent. The sleeves are then bonded to the balloon ends with a polyurethane adhesive or the like or by known welding techniques. Otherwise, the sleeves may be held in place via elastic tension.

The thickness of the sleeves, at the point of abutment with the ends of the stent, may be less than that of the stent ends, but preferably is approximately the same to provide a smooth transition from the sleeve to the stent.

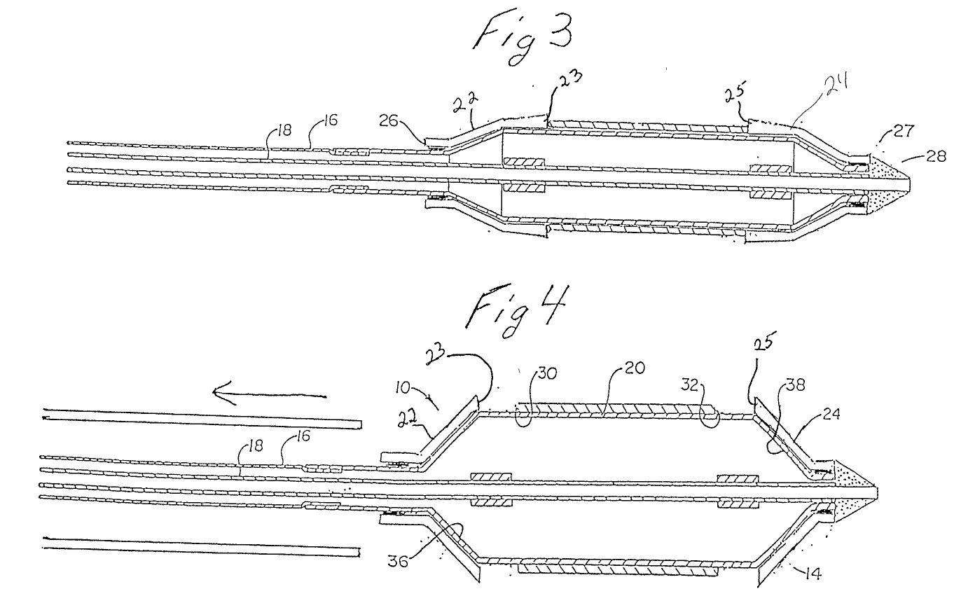

In other embodiments, as seen in FIGS. 3-6, the end of the sleeves 23, 25 may be increased in thickness to provide a more secure “seat” for the stent. This increase in thickness provides greater prevention of axial movement of the stent and assures maximum contact with the ends of the stent without overlapping them.

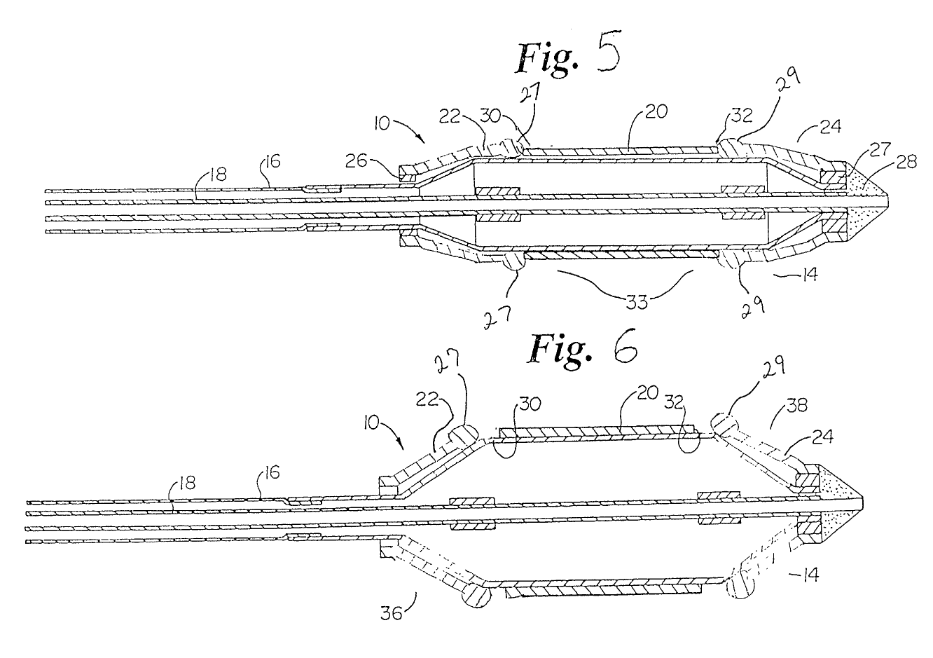

FIGS. 3-4 show sleeves 22,24 that increase gradually in thickness at the ends 23,25 abutting the ends of the stent. FIGS. 5-6, illustrate the increase in thickness by incorporating a bulge-like ring 31, 29 around the circumference of the end of the sleeves. The ends have increase thickness and rigidity integral with the sleeves themselves. The actual dimensions are not critical to the invention. The end of the sleeves need only abut the ends of the stent. However, as mentioned above, if a low profile is needed, the sleeves of the preferred embodiment are as shown in FIGS. 1-2.

As mentioned above, although two sleeves are preferable, embodiments of the present invention include having one sleeve at either end.

Other embodiments are within the claims to this invention. For example, referring to FIGS. 7-10 , the sleeves 22, 24, seen in FIGS. 1 and 2 may take the form of wire coils 24b, 24c which may for example be stainless steel, nitinol or polyamides such as nylon, or other plastics such as polyethylene or polyimide material or the like. The spiral may be cut only partially into the body as a spiral cut or it may be cut all the way through, as shown. The coil configuration aids in the urgence of the sleeve against the ends of the stent. FIGS. 7-10 only show one side of the catheter balloon. It should be understood that the mirror sleeve is similarly constructed.

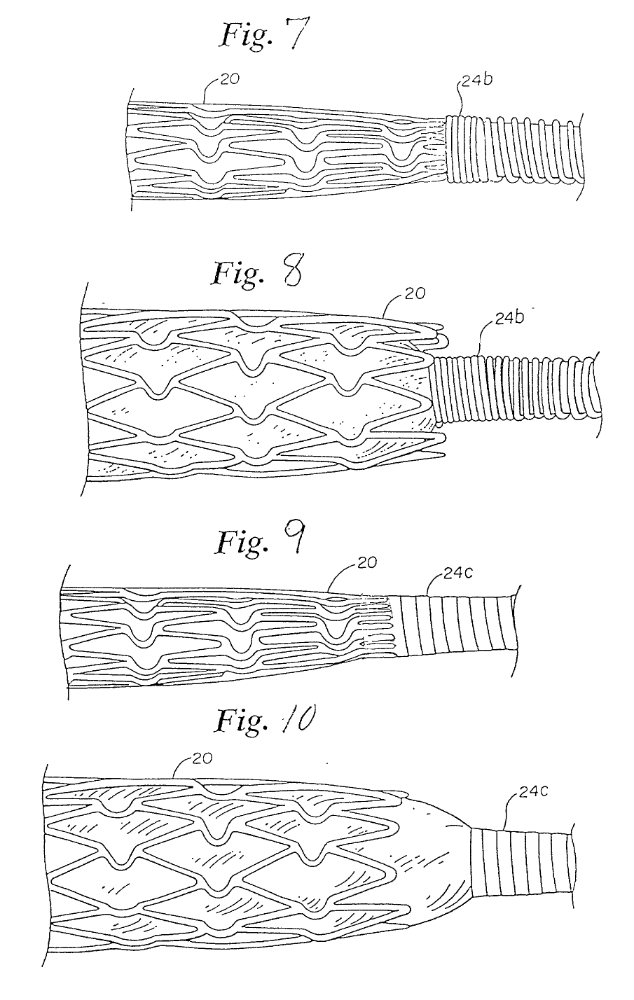

FIG. 7 shows a collapsed balloon with a coil sleeve abutting the end of the stent. FIG. 8 shows and expanded balloon and the release of the stent. FIGS. 9-10 are similar illustrations, save the coil is made from a flatter ribbon-like material. In both cases, one end of the sleeve is connected to the catheter and the other end abuts the end of the stent. Once again, only one end is shown, however, both ends may incorporated a sleeve.

Axial rigidity of the sleeves may be altered depending on the type of retraction desired. The sleeves can be configured in a number of ways for various desired retraction modes. For example, in the stent expansion sequence, i.e. middle first or ends first, or in sleeve retraction. In sleeve retraction, the rigidity of the sleeves may be adjusted to allow the sleeve to stay on the cones during expansion of the balloon aiding re-wrap of the balloon, as shown in the figures, or fold back onto themselves toward the waist of the balloon. More rigid material will tend to retract off the cones as well as allow the middle of the stent to expand first.

The invention also contemplates an embodiment without a stent. The sleeves aid in rewrap of the balloon after use of the balloon.

The invention also contemplates an embodiment wherein the stent ends expand prior to expansion of the middle of the stent. In such a case, lower strength sleeves may be used to allow the prior expansion of the ends of the stent and the balloon body.

While the invention has been described in connection with what is presently considered to be the most practical and preferred embodiments, it is to be understood that the invention is not to be limited to the disclosed embodiments but, on the contrary, is intended to cover various modifications and equivalent arrangements included within the spirit and scope of the appended claims.

The above examples and disclosure are intended to be illustrative and not exhaustive. These examples and description will suggest many variations and alternatives to one of ordinary skill in this art. All these alternatives and variations are intended to be included within the scope of the claims, where the term “comprising” means “including, but not limited to”. Those familiar with the art may recognize other equivalents to the specific embodiments described herein which equivalents are also intended to be encompassed by the claims. Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below (e.g. claim 3 may be taken as alternatively dependent from claim 2; claim 5 may be taken as alternatively dependent on claim 2, claim 3 or claim 4; claim 12 may be taken as alternatively dependent from claim 11; etc.).

What is claimed is as follows:

1. A stent delivery system comprising:
a catheter comprising an expandable distal portion, having an outer diameter, constructed and arranged for expanding the outer diameter of said expandable distal portion from a contracted state to an expanded state;
a stent positioned around said distal portion of said catheter, said stent having a contracted condition and being expandable to an expanded condition, the stent being sized in said contracted condition to surround said expandable distal portion in its contracted state, said stent having a first end and a second end, wherein at least a portion of the stent may be positioned over a portion of said expandable portion of said catheter; and
a first sleeve in the region of said distal portion of said catheter positioned around said catheter, the first sleeve having a first end attached to said catheter and a second end, said second end of the first sleeve abutting the first end of the stent.

2. The stent delivery system of claim 1, wherein the stent is expanded by expansion of said balloon and the stent is mounted on the balloon.

3. The stent delivery system of claim 1, further comprising a second sleeve in the region of said distal portion of said catheter positioned around said catheter, the second sleeve having a first end attached to said catheter and a second end, said second end of the first sleeve abutting the second end of the stent.

4. The stent delivery system of claim 1, wherein the second end of the first sleeve has a thickness and the first end of the stent has a thickness similar to that of the second end of the first sleeve, such that profile transition from the second end of the first sleeve and the first end of the stent is relatively smooth.

5. The stent delivery system of claim 3, wherein the second end of the first sleeve has a thickness and the first end of the stent has a thickness similar to that of the second end of the first sleeve, such that profile transition from the second end of the first sleeve and the first end of the stent is relatively smooth.

6. The stent delivery system of claim 1, said first sleeve being formed in the shape of a thin-walled tube.

7. The stent delivery system of claim 1, further comprising an outer

8. The stent delivery system of claim 3, said sleeves being formed from polyurethane.

9. The stent delivery system of claim 3, said sleeves being formed from any elastomer able to be expanded with a balloon angioplasty catheter, and formable into a thin-walled tube.

10. The stent delivery system of claim 1, wherein the second end of the first sleeve has an annular region of increased thickness.

11. The stent delivery system of claim 3, wherein the second ends of the sleeves have an annular region of increased thickness.

12. The stent delivery system of claim 10, wherein the annular region of increase thickness is in the form of a bulbous bulge.

13. The stent delivery system of claim 11, wherein the annular regions of increase thickness are in the form of a bulbous bulge.

14. The stent delivery system of claim 1, said first sleeve being formed in the shape of a coil, such that the second end of the first sleeve is urged against the first end of the stent.

15. The stent delivery system of claim 3, said sleeves being formed in the shape of a coil, such that the second ends of the sleeves are urged against the ends of the stent.

16. The stent delivery system of claim 1, said second end of said first sleeve having greater thickness relative to the first end of the first sleeve.

17. The stent delivery system of claim 3, said second ends of said sleeves having greater thickness relative to the first ends of the sleeves.

18. The stent delivery system of claim 1, wherein the second end of the first sleeve does not overlap the first end of the stent.

19. The stent delivery system of claim 3, wherein the second ends of the sleeves do not overlap the ends of the stent.

20. The stent delivery system of claim 3, wherein the second ends of the sleeves are urged against the ends of the stent, whereby when said expandable distal portion is expanded from said contracted state to said expanded state said stent is expanded, the center portion of the stent being expanded first while the expansion of the ends of the stent are delayed until the release force between the second ends of the sleeves and the ends of the stent are overcome by the expansion of the expandable distal portion, thereby providing an overall uniform expansion of the stent.
